(19) United States
(12) Patent Application Publication
(10) Pub. No.: US 2008/0108965 A1
(43) Pub. Date: May 8, 2008

(54) Sanitary Diaper Changing Kit

(75) Inventors: William Michael Christensen, Westlake, OH (US); Thomas George Petredis, Amherst, OH (US); Grant Logan, Lorain, OH (US)

Correspondence Address: RANKIN, HILL & CLARK LLP, 925 EUCLID AVENUE, SUITE 700, CLEVELAND, OH, 44115-1405, US

(73) Assignees: William Michael Christensen, Westlake, OH (US); Grant Logan, Lorain, OH (US)

(21) Appl. No.: 11/548,432

(22) Filed: Oct. 11, 2006

Related U.S. Application Data
(60) Provisional application No. 60/726,834, filed on Oct. 17, 2005.

(51) Int. Cl. A61F 13/15

(52) U.S. Cl. 604/385.06; 206/570

(57) ABSTRACT

A sanitary diaper changing kit includes a package, a pair of disposable gloves in the package, and a re-sealable disposal bag in the package. Alternatively, the kit may include other materials such as small single-use package of moist wipes or towelettes, a small single-use package of antiseptic wipes, a small single use package of petroleum jelly and a small single-use package of powder. The sanitary diaper changing kit may be packaged and sold with a disposable diaper, or may be sold separately from diapers to be used in conjunction with a disposable diaper. The kit allows the person changing the diaper to be isolated from the contaminated diaper during the diaper changing operation and provides a convenient means by which the soiled diaper and other used or soiled materials may be disposed of in a sanitary procedure and diaper wearer remains isolated from care giver.

### FIELD OF INVENTION

The present invention relates to changing diapers, and more particularly, to a diaper changing kit containing a unique combination of elements useful during the diaper changing operation.

### BACKGROUND OF THE INVENTION

In the process of changing a diaper involves many operations in addition to simply removing the soiled diaper and replacing it with a clean one. Often, the diaper wearer must be cleaned, and wet wipes are often used for this process. Baby powder and ointment or petroleum jelly often must be applied. When diapers are changed at home, a changing table is usually set up with the necessary elements at hand. However, when changing diapers outside the home, no such facilities are usually available, so the person changing the diaper might carry a diaper changing bag with all of the necessary supplies. This bag is usually difficult to carry and sometimes may be left behind. In addition, all of the supplies must be unpacked from the bag for each changing and must be repacked into the bag thereafter.

Various attempts have been made to provide accessories near at hand during the diaper changing process. For example, in U.S. Pat. No. 4,221,221, issued to Ehlich, a disposable diaper is provided with a packet of powder, a packet containing a disposable towel and a packet containing baby oil attached to the diaper, so that they can be removed and used during the diaper changing. In U.S. Pat. No. 4,702,378 issued to Finkel et al., a diaper changing kit is provided which contains a diaper and baby care products in a wrapper which can be reused to dispose of the soiled diaper. In U.S. Pat. No. 5,443,161 issued to Jonese, a kit comprising two diapers, two disposal bags, two wet wipers, baby powder and baby cream is provided. In U.S. Pat. No. 6,723,080, issued to Habib et al., a diaper changing kit is provided containing a disposable diaper, and one or two packages of wipes along with a pre-folded liner on which the baby can be changed, a package of hand sanitizer and a disposal bag. However, none of these kits have been satisfactory because they do not provide means for isolating the person changing the diaper from the contaminated materials.

Diapers have usually been changed without adequate sanitary protection, because the person changing the diaper is not isolated in any way from the soiled diaper and its contents. Human waste contains many bacteria and other microorganisms that can be extremely dangerous. In addition, coming into contact with human waste is generally repugnant and something that people try to avoid as much as possible. No isolation from the soiled diaper has been provided during the changing process or during the process of cleaning and re-diapering the baby or during the process of discarding the soiled diaper and all materials used in the diaper changing operation.

The diapers and the materials needed to isolate a care-giver from potentially contaminated substances have been sold separately and inconveniently. This has not facilitated a sanitary process of changing diapers, and in turn, has inhibited the routine prevention of disease.

### SUMMARY OF INVENTION

The present invention provides a solution to these problems by providing a unique diaper changing kit that overcomes the difficulties of the prior art.

The sanitary diaper changing kit of the present invention makes it extremely easy to avoid unsanitary and unsafe diaper changing by conveniently providing protective gloves and a sealable refuse bag in a handy package which may be kept and used with each new disposable diaper. The package may optionally include other diaper changing materials, such as antiseptic wipes, moist wipes, petroleum jelly and protective powder. Further, the package may optionally include a new diaper. By having the sanitary diaper changing kit conveniently placed with each new diaper, care givers can quickly and properly protect themselves and others while changing soiled diapers.

The sanitary diaper changing kit of the present invention effectively prevents the spread of disease by providing the proper materials for changing every diaper in a sanitary manner and for discarding soiled and contaminated diapers and related materials without contaminating other persons or objects. By having protective gloves available and ready for use, the person changing the diaper is protected from the soiled diaper, and the diaper wearer is protected from persons changing diapers who might have any infectious or contaminated substance on their hands. The convenience of having these protective materials included with every new diaper allows the person changing the diaper to readily ensure the diaper changing procedure is accomplished in an effective sanitary manner. The convenience and compact nature of the kit also facilitates effective sanitary diaper changing at home, while traveling in cars, on airplanes, on buses and on boats, at public functions, at private functions, while shopping, and at restaurants, and even for health care providers in hospitals, hospices, nursing homes and day care centers.

A major advantage of routinely using the sanitary diaper changing kit of the present invention is establishing and maintaining a more sanitary and healthier environment for care-givers, diaper-wearers and others coming into contact with the diaper-wearer and the care-giver. Using the sanitary diaper changing kit of the present invention, the materials that are needed to protect all related persons from being contaminated by human waste during the diaper changing process are readily and conveniently available with each new diaper. There is no need to have separate containers of the necessary items. All of the required materials are readily accessible by the person changing the diaper. The present invention eliminates worries of forgetting to pack or running out of needed materials.

The present invention also provides two important elements that are often not included in diaper changing supplies—protective gloves and a sealable refuse bag. The addition of these materials assures that the diaper changing process will be completed in the most sanitary manner possible. The diaper changer is protected by first donning the protective gloves so that he or she is fully isolated from the soiled diaper and diaper wearer. The protective gloves are disposable, so that there is no need to clean or otherwise prepare the gloves after use. In addition, the sealable refuse bag provides a means by which all of the used materials including the soiled diaper and the disposable protective gloves may be disposed of in as sanitary a process as possible. Using the sanitary diaper changing kit of the present invention will effectively eliminate the contamination between the diaper wearer and the person changing the diaper.

The convenience of having a diaper prepackaged with the necessary gloves and refuse bag, along with wipes, petroleum jelly and powder, drastically reduces the amount of materials that need to be carried about when traveling, visiting or otherwise going outside the home. It also provides added convenience if a diaper needs to be changed in another part of the home other than the normal diaper changing location where the diaper changing supplies are stored. The benefit of this convenience is amplified when changing diapers in close quarters where room and counter space are limited. There is no need to have bottles, tubes and boxes set up all around the person changing the diaper to accomplish the same effective and sanitary diaper changing process that can be done with the sanitary diaper changing kit of the present invention. Also, by properly using the disposable protective gloves, the refuse bag and optionally the other changing materials provided in the sanitary diaper changing kit of the present invention, there is no contamination of the large containers of changing materials ordinarily used when changing diapers.

Besides facilitating a healthier, more sanitary diaper-changing process and being more efficient, the sanitary diaper changing kit of the present invention actually allows for a faster changing process, by organizing all of the required diaper changing materials and having them all at the point of use, every time

These and other advantages are provided by the present invention of a sanitary diaper changing kit comprising a package, a pair of disposable gloves in the package, and a re-sealable disposal bag in the package.

### BRIEF DESCRIPTION OF THE DRAWINGS

FIG. 1 is a perspective view of a basic sanitary diaper changing kit according to the present invention.

FIG. 2 is a perspective view similar to FIG. 1 of an alternative embodiment of the diaper changing kit of the present invention.

FIG. 3 is a perspective view similar to FIGS. 1 and 2, showing the diaper changing kit of FIG. 1 packaged with a disposable diaper.

### DETAILED DESCRIPTION OF PREFERRED EMBODIMENTS

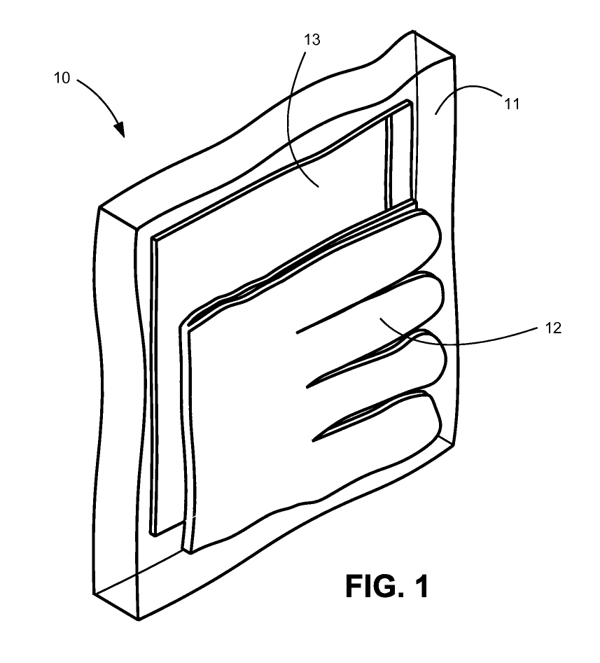

Referring more particularly to the drawings, and initially to FIG. 1, there is shown the sanitary diaper changing kit 10 of the present invention. The kit comprises a package 11 which may be clear as shown or may be opaque. The package 11 is preferably made of a plastic film, such as polyethylene.

Inside the package 11, the kit 10 comprises a pair of disposable gloves 12 and a re-sealable plastic disposal bag 13. The gloves 12 are preferable of the one-size-fits-all type made of polyethylene and typically worn in food handling, and are folded as shown so that the overall size of the package is minimized 11. The gloves 12 may also be latex gloves such as those commonly worn by health care workers; however, latex gloves are more difficult to put on than the looser food service gloves, and the added dexterity provided by latex gloves is usually unnecessary in the diaper changing process in most instances. The disposal bag 13 may be of the type used to store food having a plastic zipper lock or other similar bags. Suitable bags are those similar to the bags sold under the trademark Ziploc. It is important that the bag be capable of being effectively sealed so that all soiled and contaminated materials can be placed in the bag are safely contained therein and so that no smell or other effects from the refuse are present.

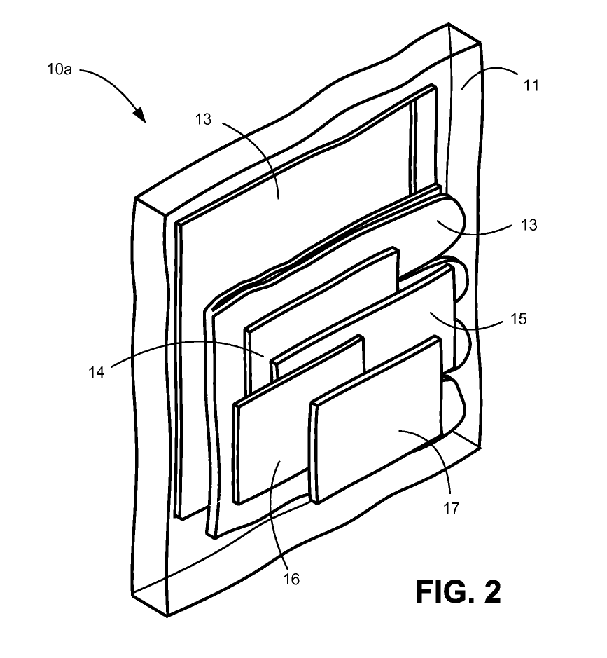

As shown in FIG. 2, the kit may also be modified to be a kit 10a that includes several other items, such as a small single-use package 14 of moist wipes or towelettes, a small single-use package 15 of antiseptic wipes, a small single use package 16 of petroleum jelly and a small single-use package 17 of powder.

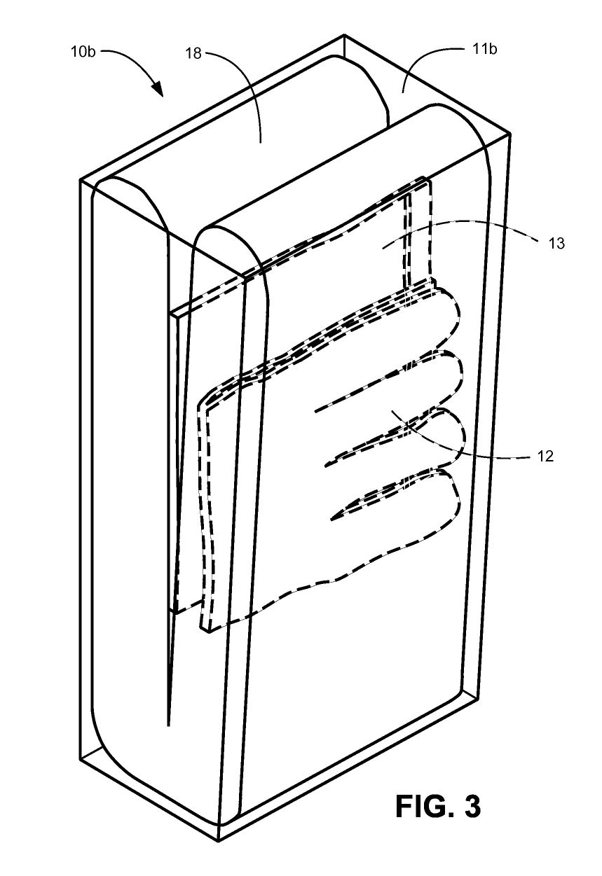

As shown in FIG. 1, the basic sanitary diaper changing kit 10 of the present invention, which includes the protective gloves and the re-sealable plastic bag for refuse may be sold separately as a stand alone kit. The kit may also be sold included and pre-packaged with a disposable diaper, as shown in FIG. 3. The modified kit 10b shown in FIG. 3 includes a larger package 11b which contains a disposable diaper 18. The pair of disposable gloves 12 and a re-sealable plastic disposal bag 13 are preferably packaged inside the folded diaper so that they can be easily removed when the diaper is unfolded. The addition of the gloves 12 and the disposal bag 13 adds little to the weight and volume of the diaper alone.

Similarly, the modified sanitary diaper changing kit 10a may be sold with a disposable diaper. In this arrangement, the gloves 12 and the disposal bag 13 would be packaged inside the folded diaper along with the single-use package 14 of moist wipes or towelettes, the single-use package 15 of antiseptic wipes, the single use package 16 of petroleum jelly, and the single-use package 17 of powder.

The diaper changing kit of the present invention is used as follows. When it is necessary to change a soiled diaper, the care giver obtains a kit at the same time that he or she obtains a new diaper, or the new diaper contains or is included with the kit. Alternatively, the care giver may obtain a diaper from a supply of conventional disposable diapers and obtain a diaper changing kit from a separate supply of such kits. The care giver opens the sanitary diaper changing kit package 11 and removes the contents, placing the contents within the care giver's reach at the changing area. The care giver can prepare all materials for quick and easy use during the diaper changing. The care giver takes the gloves 12 and puts them on. The care giver can then begin removing the soiled diaper, making sure that only one of the gloved hands is used to touch any contaminants and leaving the other gloved hand clean. The care giver places the soiled diaper into the supplied refuse bag 13. If the kit is provided with the other packages, the care giver can use the contents of those packages to further clean the diaper wearer, and the care giver then puts the empty or partially used packages into the refuse bag. The care giver then removes one of the gloves and places it into the refuse bag. The care giver can now apply the new diaper and perform any other necessary tasks, such as applying lotion or powder. The diaper wearer will continue to be isolated from the care giver because the care giver can use the hand that is still wearing a glove to touch the diaper wearer; the other ungloved hand can remain isolated. When all tasks are completed, the care giver removes the remaining glove, puts it into the refuse bag, seals the bag closed using the plastic zipper on the refuse bag, and properly disposes of the sealed refuse bag. All of the unsanitary and soiled articles along with all of the discarded articles are now safely sealed away in the refuse bag, and will not contaminate any further activities. This process very effectively inhibits the spread of disease by isolating the care giver from contamination from the soiled diaper, and isolating the diaper wear from any contamination from the care giver.

It can be seen that use of the sanitary diaper changing kit of the present invention prevents the spread of disease to the diaper changer, the diaper wearer and any other individuals that might result from changing a soiled diaper by isolating the person changing the diaper from the soiled and contaminated diaper and by isolating the diaper wearer from the hands of the person changing the diaper. Using the sanitary diaper changing kit of the present invention, this is accomplished routinely, conveniently and effectively. The sanitary diaper changing kit also provides a safe, sealable receptacle for use in discarding soiled diapers and contaminated changing materials.

Besides facilitating a healthier, more sanitary diaper-changing process and being more efficient, the sanitary diaper changing kit of the present invention actually allows for a faster changing process, by organizing all of the required diaper changing materials and having them at the point of use, every time

While the sanitary diaper changing kit of the present invention has special application for use with changing baby diapers, it can also be used in other applications. The invention can be used in conjunction with changing adult diapers. It can be used at home, away from home, at a hospital and hospice or anywhere the need arises. The invention can also be modified for other uses such as providing a prepackaged kit having contents for use in first aid, where the protective gloves, antiseptic wipes and sealable refuse bag are effective in protecting the caregiver, the first aid victim and others in proximity. It can also be used in situations in which there is no access to conventional toilet facilities, such as in disaster relief, camping, and similar situations.

It should be realized that the embodiment described herein is only representative of the invention and is not intended to limit the invention to one particular embodiment as the invention includes all embodiments falling within the scope of the appended claims. Additional advantages and modifications will readily occur to those skilled in the art. Therefore, the invention in its broader aspects is not limited to the specific details and illustrative examples shown and described herein. Accordingly, various modifications may be made without departing from the spirit or scope of the general inventive concept as defined by the appended claims and their equivalents.

What is claimed is:

1. A sanitary diaper kit comprising:
a package;
a pair of disposable gloves in the package; and
a re-sealable disposal bag in the package.

2. The diaper changing kit of claim 1, comprising in addition a disposable diaper in the package.

3. The diaper changing kit of claim 1, comprising in addition a single-use container of moist wipes in the package.

4. The diaper changing kit of claim 1, comprising in addition a single-use container of antiseptic wipes in the package.

5. The diaper changing kit of claim 1, comprising in addition a single use container of petroleum jelly in the package.

6. The diaper changing kit of claim 1, comprising in addition a single-use container of powder in the package.

7. The diaper changing kit of claim 1, wherein the re-sealable bag is made of polyethylene.

8. The diaper changing kit of claim 1, wherein the re-sealable bag has a zipper locking strip.

9. The diaper changing kit of claim 1, wherein the disposable gloves are made of polyethylene.

10. A sanitary diaper kit comprising:
a package;
a pair of disposable polyethylene gloves in the package;
a re-sealable polyethylene disposal bag in the package, the bag having a resealing zipper locking strip,
a disposable diaper in the package.
a single-use container of moist wipes in the package.
a single-use container of antiseptic wipes in the package.
a single use container of petroleum jelly in the package.
a single-use container of powder in package.

11. A method for changing a diaper on a wearer, comprising the steps of:
obtaining a diaper changing kit comprising a package, a pair of disposable gloves in the package; and a re-sealable disposal bag in the package;
donning the disposable gloves from the diaper changing kit;
removing a soiled diaper from the wearer;
putting the soiled diaper into the disposal bag from the diaper changing kit;
putting a clean diaper on the wearer;
removing the disposable gloves;
putting the disposable gloves into the disposal bag; and
discarding the disposal bag.

12. The method for changing a diaper of claim 11, wherein the diaper changing kit contains a single-use container of moist wipes in the package, comprising the additional step of using the moist wipes after removing the soiled diaper and before putting the clean diaper on the wearer.

13. The method for changing a diaper of claim 11, wherein the diaper changing kit contains a single-use container of petroleum jelly in the package, comprising the additional step of applying the petroleum jelly on the diaper wearer after removing the soiled diaper and before putting the clean diaper on the wearer.

14. The method for changing a diaper of claim 11, wherein the diaper changing kit contains a clean diaper, and the step of putting the clean diaper on the wearer is performed using the clean diaper from the diaper changing kit.

15. The method of changing a diaper of claim 11, comprising the additional steps of removing one of the pair of disposable gloves after removing the soiled diaper and before putting the clean diaper on the wearer, and removing the other of the pair of disposable gloves are purring the clean diaper on the wearer.
